(19) United States
(12) Patent Application Publication
(10) Pub. No.: US 2013/0123889 A1
(43) Pub. Date: May 16, 2013

(54) Apparatus for Preventing Motion Sickness and Related Method

(71) Applicants: Jay W. Katz, Palm Coast, FL (US); Gunnar H. Hildemann, Palm Coast, FL (US)

(72) Inventors: Jay W. Katz, Palm Coast, FL (US); Gunnar H. Hildemann, Palm Coast, FL (US)

(21) Appl. No.: 13/728,914

(22) Filed: Dec. 27, 2012

Related U.S. Application Data
(63) Continuation-in-part of application No. 12/750,358, filed on Mar. 30, 2010, now abandoned.

(51) Int. Cl. A61F 7/12

(52) U.S. Cl. CPC A61F 7/12
USPC 607/105

(57) ABSTRACT

The invention is directed toward a method of reducing motion sickness comprising the steps of introducing cooled gas into a user's ear canal to reduce the temperature of the user's tympanic membrane and vestibular system. The gas is circulated in the user's inner ear, and then exhausted to the atmosphere. The cooled gas induces nystagmus in the user, promotes vestibular desensitization and resistance to motion sickness. The apparatus contemplated for the prevention of motion sickness has an earpiece having a conduit that dispenses cooled gas into the user's ear canal. Gas is provided by a gas source connected to the earpiece with flexible tubing.

### CROSS-REFERENCE TO RELATED APPLICATION

This is a Continuation-in-Part Application of U.S patent application Ser. No. 12/750,358 filed on Mar. 30, 2010 entitled “Method for Relieving Motion Sickness and Related Apparatus,” the contents of which are incorporated by reference herein.

### FIELD OF THE INVENTION

The present invention is generally directed toward an apparatus and related method for cooling a user's tympanic membrane to promote relief from motion sickness.

### BACKGROUND

Motion sickness, also known as kinetosis, is a medical condition caused by a conflict between visually perceived stimuli and the vestibular system's sense of movement. Put another way, motion sickness is generally caused by the difference between how an individual's eyes perceive movement in comparison to how the inner ear senses the same stimuli. Motion sickness, depending upon the underlying cause, is also commonly referred to as seasickness, car sickness, or air sickness.

Studies indicate that over ⅓ of the population is susceptible to motion sickness in mild circumstances such as from merely being on a boat in calm water. These same studies find that over ⅔ of the population are susceptible to motion sickness in more severe conditions. Individuals suffering from motion sickness typically exhibit a variety of symptoms including dizziness, fatigue, and nausea. In cases of prolonged motion sickness, subjects may even vomit (with little symptom relief).

In a related condition known as sophite syndrome, subjects may experience motion-induced fatigue, yawning, and sleep disturbances as a result of incongruous visual and vestibular stimulation.

There are a number of methods typically employed to treat motion sickness. Drug treatment options include medications such as dimenhydrinate (Dramamine®) and meclizine (Bonine® or Antivert®). Transdermal patches that include a quantity of scopolamine have also been used as a treatment option. General pharmacological treatments for nausea and vomiting can also be employed to treat minor conditions. Anti-histamines, such as promethazine, can also improve minor motion sickness, although they commonly cause side effects such as drowsiness. While many of the pharmacological options provide relief from motion sickness, they require ingestion of chemicals which impose the risk of possible side effects such as drowsiness and loss of attention. Additionally, pharmacological options may hinder one from performing certain tasks. For example, taking certain medications while engaging in occupational activities, such as, for example, piloting aircraft, seafaring vessels, motor vehicles, and heavy equipment may be contraindicated.

NASA has performed extensive research on the use of electric forms of treatment for motion sickness. Over half of NASA astronauts have reported some form of motion sickness while in space flight. The most severe reports of such “space sickness” come during an astronaut's maiden flight. A treatment option developed by NASA for those suffering from motion sickness is a form of eyeglasses having liquid crystal display (LCD) shutter lenses that create stroboscopic vision.

Other treatments include having a person experience car sickness fixate on a stationary object while in a moving vehicle. One example includes looking out of the window of a moving vehicle and gazing toward the horizon, which helps re-orient a sense of balance. Conversely, having an individual close his or her eyes is another treatment option. Napping or sleeping can also help offset the symptoms of motion sickness. Chewing gum has also been reported to improve car sickness symptoms. These treatment options typically fail to provide complete or lasting relief. Moreover, none of these options typically provides a permanent solution to severe conditions.

Accordingly, there is a need for a simple, yet more efficacious means to treat motion sickness. An ideal apparatus for such treatment should be portable, reusable, and non-toxic. Additionally, there is a need for a related method of preventing motion sickness—especially in individuals averse to taking anti-motion sickness medications. The apparatus and related method of treatment should be robust and provide long-term relief even for severe conditions.

### SUMMARY OF THE INVENTION

In view of the foregoing background, it is therefore an object of the present invention to provide a non-pharmacological, portable, reusable, and non-toxic apparatus and related method for decreasing sensitivity to and relieving the symptoms of motion sickness that is quickly and easily self-administered. Specifically, through gas-cooling a user's inner ear, the confusion between vestibular organs and visually perceived movement that promotes motion sickness is attenuated.

In particular, the invention contemplates an apparatus for the prevention of motion sickness that comprises a gas supply and a conduit defining a first opening attachable to the gas supply and also having a second opening disposed proximate a user's ear canal to allow a gas introduced into the conduit from the gas supply to flow into the user's ear canal and cool the user's tympanic membrane.

In a related embodiment, an earpiece that is sized and dimensioned to engage a user's ear has a protrusion that projects proximate the user's ear canal. The conduit passes through the protrusion, and the second opening is disposed proximate a distal end of the protrusion and the first opening is proximate the user's outer ear. An exhaust port is defined by the earpiece that provides an exit route for the gas introduced into the user's ear canal so to reduce pressure within the user's ear canal that may be uncomfortable.

The gas supply is a cylinder of compressed gas, or in a related embodiment, an air pump. The gas is preferably carbon dioxide, or in a related embodiment, air. Gas mixtures are also contemplated.

In one embodiment of the invention, a filter is disposed in-line with the conduit, between the gas supply and the earpiece, to prevent debris from entering the user's ear canal. The filter may comprise a fibrous matrix. The filter may, in a related embodiment, comprise a desiccant.

In yet another related embodiment, a venturi valve is disposed in-line with the conduit, between the gas supply and the earpiece, to aid in regulating temperature and flow rate of the gas introduced into the user's ear canal.

The gas introduced into the conduit comprises a flow rate between about 3 L/min and 5 L/min, and a temperature between about 60° F. of and about 75° F.

The invention also contemplates a related method of reducing motion sickness. The method comprises the steps of introducing gas into a user's ear canal; cooling a user's tympanic membrane with the gas; exhausting the gas from the user's ear canal; and inducing nystagmus in the user.

The gas is introduced into the user's ear canal through an earpiece worn by the user. The gas is air or alternatively carbon dioxide. In one embodiment the method comprises the step of filtering the gas before the gas is introduced into the user's ear canal. A related embodiment comprises the step of reducing the velocity of the gas before the gas is introduced into the user's ear canal. In yet another related embodiment, the method comprises the step of desiccating the gas before the gas is introduced into the user's ear canal.

The method of reducing motion sickness optionally further comprises the step of passing the gas through a venturi valve before the gas is introduced into the user's ear canal.

The method further comprises the step of maintaining a flow rate of the gas between about 2 L/min and about 12 L/min, and preferably between about 3 L/min and 5 L/min. The method also comprises the step of maintaining the gas at a temperature of between about 50° F. and about 78° F., and preferably between about 60° F. and about 75° F.

The gas is introduced into the user's ear canal for between about 1 minute and about 20 minutes per session, and preferably between about 2 minutes and about 5 minutes. In a related embodiment of the method of reducing motion sickness, the gas is introduced into the user's ear canal 1 time per day for 1 to 10 consecutive days.

An alternate method of reducing motion sickness comprises the steps of: introducing a carbon dioxide gas mixture into a user's ear canal through an earpiece worn by the user, exhausting the gas from the user's ear canal, maintaining gas circulation in the user's ear canal for between about 2 minutes and about 5 minutes, maintaining the gas at a temperature of between about 60° F. and about 75° F., maintaining a flow rate of the gas between about 3 L/min and 5 L/min, cooling a user's tympanic membrane with the gas, and inducing nystagmus in the user. This alternate method may be repeated 1 time per day until the symptoms associated with motion sickness are reduced in the user.

### BRIEF DESCRIPTION OF THE DRAWINGS

For a fuller understanding of the invention, reference is made to the following detailed description, taken in connection with the accompanying drawings illustrating various embodiments of the present invention, in which:

FIG. 1 illustrates the various components of outer, middle and inner ear and one embodiment of an earpiece in a used state;

FIG. 3 illustrates the earpiece of FIG. 1, further illustrating a path of gas flow; and

FIG. 2 is a diagram illustrating the central components one embodiment of the invention.

### DETAILED DESCRIPTION OF EMBODIMENTS OF THE INVENTION

In the Summary of the Invention above and in the Description of Embodiments of the Invention, reference is made to particular features (including method steps) of the invention. It is to be understood that the disclosure of the invention in this specification includes all possible combinations of such particular features, regardless of whether a combination is explicitly described. For example, where a particular feature is disclosed in the context of a particular aspect or embodiment of the invention, that feature can also be used, to the extent possible, in combination with and/or in the context of other particular aspects and embodiments of the invention, and in the invention generally.

The term “comprises” is used herein to mean that other features or steps are optionally present. When reference is made herein to a method comprising two or more defined steps, the steps can be performed in any order or simultaneously (except where the context excludes that possibility), and the method can include one or more steps which are carried out before any of the defined steps, between two of the defined steps, or after all of the defined steps (except where the context excludes that possibility).

This invention may be embodied in many different forms and should not be construed as limited to the embodiments set forth herein. Rather, these embodiments are provided so that this disclosure will be thorough and complete, and will convey the scope of the invention to those skilled in the art. Other related embodiments will be known and understood by those of ordinary skill in the art now having the benefit of the teachings of the present invention.

Embodiments of the invention are described herein in connection with an apparatus for cooling a user's tympanic membrane. It is to be understood, however, that the invention is not limited to the specific sizes or shapes described.

The present invention relates to an apparatus and related method of preventing motion sickness through cooling a user's vestibular organs. The apparatus introduces a cooled gas into a user's ear canal so that the tympanic membrane is cooled. The method is directed to administering gas-mediated cooling of a user's tympanic membrane and inducing habituation of the vestibular system to prevent or reduce the adverse effects of activities that may induce motion sickness, loss of balance, and vertigo.

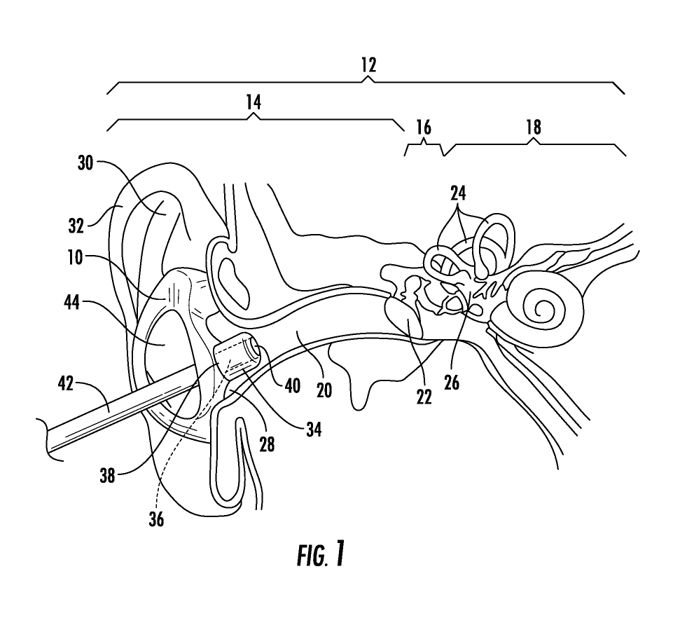

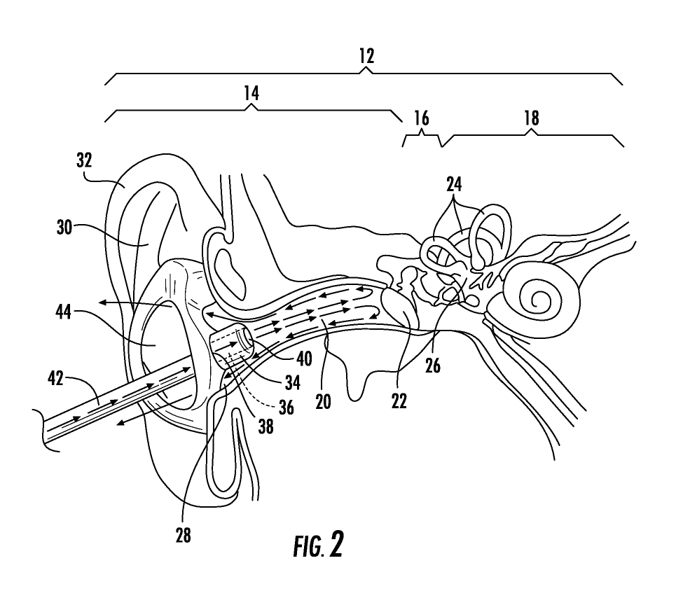

FIG. 1 illustrates one embodiment of an earpiece 10 contemplated by the invention as well as the primary portions of the human vestibular-auditory system 12. As shown in FIG. 1, there are three primary components of the human vestibular auditory system 12: the outer ear 14, middle ear 16 and inner ear 18. The outer ear 14 begins from outside of the head and extends through the ear canal 20 and ends at the tympanic membrane 22 (more commonly known as the ear drum). The middle ear 16 contains organs that function predominantly for receiving auditory stimulus. The inner ear 18 organs aid in auditory processing, yet additionally comprises the organs of the vestibular system.

The vestibular system provides input for an individual's sense of balance, motion, and position. It comprises three semi-circular canals 24 connected to the vestibule 26. Structures in the semicircular canals 14 and vestibule 26 (hair cells and otolith organs respectively, not illustrated) detect head movement, head rotation, and help sense gravity, position, and acceleration.

Upon vestibular stimulation due to physiologically nonconventional body movement (such as that experienced while on a rocking boat) neuronal signals may begin to differ from signals generated from visual perception, conflicting with vestibular perception and brain pathways, which can result in dizziness and nausea commonly classified as motion sickness. It is the focus of the present invention to provide a method and related apparatus for gas cooling of the vestibular system to attenuate the adverse symptoms of motion sickness due to conflicting visual, vestibular, and brain perception.

### The Earpiece

With continuing reference to FIG. 1, one embodiment of an earpiece 10 contemplated by the present invention is illustrated. The primary function of the earpiece 10 is to deliver a sufficient quantity of cooled gas to the tympanic membrane. The earpiece 10 is of a size and dimension to engage a user's outer ear 14. As illustrated, the earpiece 10 is worn by contacting the contours of the acoustic meatus 28. In a preferred embodiment, the earpiece 10 is dimensioned to facilitate handling by a user's fingers to promote comfortable insertion and positioning with the outer ear 14.

Other earpiece 10 designs are also contemplated, such as, by way of example, a substantially external earpiece 10 that rests predominantly on top of the outer ear 14 so that the earpiece 10 rests upon the medial surface of the Scaphoid fossa 30 and wraps around the Helix 32, wherein projections therefrom extend proximate to or into the ear canal 20. Other earpiece designs are also contemplated.

The earpiece 10 has a protrusion 34 that extends into the ear canal 20. The protrusion 34 has a conduit 36 that passes therethrough. Gas enters the conduit 36 through a first opening 38. The conduit 36 allows gas to exit through a second opening 40 and into the ear canal 20. The first opening 38 is sealedly attached to tubing 42 into which a gas is introduced.

The earpiece 10 also has an exhaust port 44 that allows gas introduced into the ear canal to escape. In an alternative embodiment, the earpiece 10 creates a sufficient seal to be firmly held in the outer ear 14, yet vented around the earpiece 10 so that gas escapes to outside of the ear canal 20.

FIG. 2 illustrates a preferred gas flow pathway of the present invention. Gas introduced into the tubing 42 passes through the conduit 36 and exits the earpiece 10 through the second opening 40 of the protrusion 34. Gas then enters the ear canal 20 and contacts the tympanic membrane 22. This cools the tympanic membrane and therefore the organs of the vestibular system. The gas is then exhausted out of the ear canal through the exhaust port 44.

The earpiece 10 is preferably made from a relatively compliant yet resilient material, such as rubber, silicone, methyl vinyl silicone, phenyl methyl silicone, platinum cure silicone, fluorocarbon elastomer, perfluroinated elastomer, acrylate, nitrile, hydrogenated nitrile butadiene rubber, and any other relatively compliant material known in the art. However, earpiece 10 material may also be chosen from harder materials such plastics, high density polyethylene, low density polyethylene, polyethylene terephthalate, polyvinyl chloride, polypropylene, polyoxymethylene, polystyrene, post-consumer resin, K-resin, epoxy resin, phenolic formaldehyde resin, stainless steel, aluminum, other metals, ceramic, and any other material known in the art.

### The Overall Apparatus

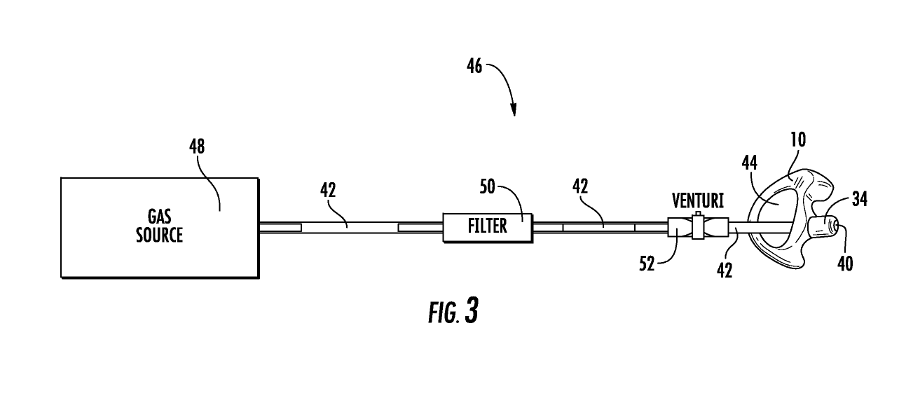

FIG. 3 is a schematic that illustrates the major components of the apparatus 46 for cooling the vestibular system as contemplated herein. Gas is introduced into tubing 42 from a gas source 48. The gas travels through the tubing 42 to pass through the earpiece 10 and into a user's ear canal 20. In the embodiment illustrated by FIG. 3, the gas passes through a filter 50 and a venturi valve 52. The apparatus 46 may have only a filter 50, only a venturi valve 52, multiple filters 50, both a filter 50 and venturi 52, or neither a filter 50 nor a venturi 52.

Tubing 52 connects the earpiece and other components of the apparatus 46. The tubing is preferably flexible conduit capable of transporting a gas therethrough. Suitable material for tubing includes, without limitation, Polyurethane, Polypropylene, polyvinyl chloride, Nylon, Polystyrene styrenic block copolymer, thermoplastic polyester elastomer, aliphatic polyether-based thermoplastic polyurethane, elastomeric thermoplastic polyurethane, polyolefins, polyamide nylon, low density polyethylene, high density polyethylene, metallocene polyethylene resins, polyether block amide, rubber, latex, nitrile, combinations thereof, and any other tubing material known in the art.

Tubing connectors 53 used to connect the earpiece 10 and other apparatus 46 components to the tubing 42 include barbed tube fittings, quick-disconnect couplers, twist-to-connect couplers, Speedfit®-type fittings, and any other tubing connector known in the art.

With continuing reference to FIG. 3, the gas source 48 provides a substantially inert gas for the apparatus 46. The gas is preferably carbon dioxide (CO₂) or air. However, gasses such as nitrogen, argon, helium, and any other medically safe and relatively inert gasses or combination of gasses known in the art are contemplated. In one embodiment, the gas source 48 is cylinder of compressed gas or liquid gas attached to the tubing 42 through a regulator. Alternatively, the gas source 48 is an air compressor or air pump. The pump is chosen from standard air pumps known in the art such as diaphragm or rotary vane pumps.

The filter 50 is disposed inline between the gas source 48 and the earpiece 10. In one embodiment, the filter 50 prevents particulate matter from passing therethrough. For example, chilled gas can cause atmospheric moisture to form ice crystals, and the filter 50 prevents these from reaching the user. In a related embodiment, the filter 50 acts as a diffuser that decreases the velocity of gas flow and/or reduces the perceived acoustic volume of the gas entering the user's ear. In yet another related embodiment, the filter contains a desiccant that reduces downstream moisture present in the apparatus 46. The apparatus 46 has a single filter 50 that accomplishes at least one of particulate filtration, diffusion, and desiccation. Alternatively, multiple filters 50 are situated in series to provide any combination of these functions.

Filters 50 contain media made from paper, bamboo, hemp fiber, activated carbon, cellulose, ethyl vinyl acetate, fibrous matrices, glass fiber media, acrylic copolymer membrane, polypropylene, hydrophobic resin bonded inorganic ceramic fiber, polyethersulfone membrane, polytetrafluoroethylene, ceramic bonded hydrophobic glass, combinations thereof, and any other filter media known in the art.

The venturi valve 52 is disposed inline between the gas source 48 and the earpiece 10, and provides two primary functions: first ambient air is introduced into the apparatus to help regulate the temperature of the gas that reaches a user's ear. For example, cooled CO₂ traveling to the earpiece 10 is warmed by the addition of ambient temperature air into the venturi valve 52 which travels into the tubing, mixing with the CO₂, so that the gas mixture that reaches the ear is warmer than CO₂ alone. A second function of the venturi valve 52 is that of flow rate regulation. The air introduced into the venturi exits the venturi valve 52 at a greater velocity than when it entered, thus increasing the flow rate of gas that reaches the earpiece 10.

### Method of Preventing Motion Sickness

The invention contemplates a method of preventing motion sickness and decreasing sensitivity to motion sickness. Reducing sensitivity of the vestibular system, and thus relieving symptoms common to motion sickness, is accomplished by using cooled gas to lower the temperature of a user's vestibular system through cooling the user's tympanic membrane and ear canal.

The volume of cooled gas is maintained at a relatively consistent volume and flow rate. A more severe condition may, however, require a longer duration or more frequent application of cooled gas exposure. The key design consideration is to have a sufficient amount and duration of cooled gas for indirectly cooling the inner ear 18, which includes, inter alia, endolymphatic fluid, octoconial crystals, and vestibular nerves.

By cooling these various portions of the vestibular system, the sensitivity of the vestibular system organs and nerves is temporarily realigned with the visual senses and brain mechanisms which create habituation and compensation. Accordingly, there exists a lower chance of confusion between the vestibular system, visual perception and central brain mechanisms, which reduces the risk and/or symptoms associated with motion sickness.

Prior to initiating treatment, it is advisable to determine that gas flow and gas temperature are within in a range comfortable to a user. Therefore, a step of the method to ensure user safety is the initial pre-use apparatus evaluation. It is necessary each time prior to use and involves releasing gas from the gas source 48 to evaluate gas volume, pressure, and temperature proximate the earpiece 10. This may be measured with instrumentation or merely approximated by placing a fingertip in the gas stream to estimate a steady and comfortable flow prior to inserting the earpiece 10 in the outer ear 14.

Another step of the method is for a user to introduce gas into their ear canal 20. Preferably, the user places the earpiece 10 of the apparatus 46 on or about their outer ear 14 so that gas flow is directed into the ear canal 20. This step includes positioning the earpiece 10 in an orientation such that cooled gas flows to the tympanic membrane 22 to ensure the gas interacts with the vestibular system. Special care should be given to prevent having the earpiece 10 from contacting the tympanic membrane 22.

Gas is introduced into a user's ear canal 20 and the temperature of the user's tympanic membrane 22 is lowered due to the presence of circulating cooled gas in the ear canal 20. As more gas is introduced into the ear canal, excess gas is exhausted from the user's ear and into the surrounding atmosphere. In a preferred embodiment, the user's tympanic membrane 22 is cooled for a duration necessary to induce nystagmus (eye movement) in the user. Cooling the tympanic membrane 22 cools the endolymphatic fluid in the semi-circular canals 24 as well as the vestibular nerves and the octoconia within the otolith organs. By cooling these various components of the vestibular system, a user may experience decreased vestibular sensitivity that reduces the risk of confusion with visually perceived motion and accordingly reduces symptoms associated with motion sickness.

In a preferred embodiment of the method, the gas introduced into a user's ear canal 20 flows at a rate maintained at between about 2 L/min and about 12 L/min, with an even more preferred gas flow rate ranging between about 3 L/min and 5 L/min.

In a preferred embodiment of the method, the gas introduced into a user's ear canal 20 is maintained at a temperature between about 50° F. and about 78° F., with an even more preferred temperature ranging between about 60° F. and about 75° F.

In a preferred embodiment of the method, gas is introduced into a user's ear canal 20 for a duration of between about 1 minute and about 20 minutes, and more preferably for between about 2 minutes and about 5 minutes.

In one embodiment of a pre-activity treatment regimen, gas is introduced into a user's ear canal 20 one time per day for one to four consecutive days. Treatment is administered prior to engaging in an activity which risks the onset of motion sickness. Such activities include, for example without limitation, riding in a car, airplane, or seafaring vessel. Treatment, however, may alternatively be increased to multiple sessions in a single day.

In one embodiment of an inter-activity treatment regimen, gas is introduced into a user's ear canal one 20 time per day in concurrence with activities that risk the onset of motion sickness. To promote maintenance of vestibular habituation, the method contemplates administering treatment for each day of a continued activity that imposes a risk of motion sickness. This will promote vestibular desensitization so that discordant visual perception does not elicit motion sickness.

1. An apparatus for the prevention of motion sickness comprising:
a gas supply;
a conduit defining a first opening attachable to the gas supply and a second opening disposed proximate a user's ear canal to allow a gas introduced into the conduit from the gas supply to flow into the user's ear canal and cool a user's tympanic membrane.

2. The apparatus for the prevention of motion sickness of claim 1 further comprising an earpiece sized and dimensioned to engage a user's outer ear, wherein the conduit passes through the earpiece, wherein the second opening is disposed proximate the user's ear canal and the first opening is disposed proximate the user's outer ear.

3. The apparatus for the prevention of motion sickness of claim 1 further comprising an earpiece sized and dimensioned to engage a user's outer ear, the earpiece having a protrusion that projects proximate the user's ear canal, wherein the conduit passes through the protrusion, wherein the second opening is disposed proximate a distal end of the protrusion and the first opening is proximate the user's outer ear.

4. The apparatus for the prevention of motion sickness of claim 2 further comprising an exhaust port defined by the earpiece that defines an exit route for the gas introduced into the user's ear canal for reducing uncomfortable pressure within the user's ear canal.

5. The apparatus for the prevention of motion sickness of claim 2 further comprising a filter connected to the conduit between the gas supply and the earpiece.

6. The apparatus for the prevention of motion sickness of claim 5 wherein the filter comprises a desiccant.

7. The apparatus for the prevention of motion sickness of claim 5 wherein the filter comprises a filter element to prevent debris from entering the user's ear canal.

8. The apparatus for the prevention of motion sickness of claim 2 further comprising a venturi valve connected to the conduit between the gas supply and the earpiece to aid in regulating temperature and flow rate of the gas introduced into the user's ear canal.

9. The apparatus for the prevention of motion sickness of claim 1 wherein the gas supply is a cylinder of compressed gas.

10. The apparatus for the prevention of motion sickness of claim 1 wherein the gas supply is an air pump.

11. The apparatus for the prevention of motion sickness of claim 10 wherein a gas in the gas supply is carbon dioxide.

12. The apparatus for the prevention of motion sickness of claim 2 wherein the gas introduced into the conduit comprises a flow rate between about 3 L/min and 5 L/min.

13. The apparatus for the prevention of motion sickness of claim 2 wherein the gas introduced into the conduit comprises a temperature between about 60° F. of and about 75° F.

14. A method of reducing motion sickness comprising the steps of:
introducing gas into a user's ear canal;
cooling a user's tympanic membrane with the gas;
exhausting the gas from the user's ear canal; and
inducing nystagmus in the user.

15. The method of reducing motion sickness of claim 14 wherein the gas is introduced into the user's ear canal through an earpiece worn by the user.

16. The method of reducing motion sickness of claim 14 wherein the gas is air.

17. The method of reducing motion sickness of claim 14 wherein the gas is carbon dioxide.

18. The method of reducing motion sickness of claim 14 further comprising the step of filtering the gas before the gas is introduced into the user's ear canal.

19. The method of reducing motion sickness of claim 14 further comprising the step of reducing velocity of the gas before the gas is introduced into the user's ear canal.

20. The method of reducing motion sickness of claim 14 further comprising the step of desiccating the gas before the gas is introduced into the user's ear canal.

21. The method of reducing motion sickness of claim 14 further comprising the step of passing the gas through a venturi before the gas is introduced into the user's ear canal.

22. The method of reducing motion sickness of claim 14 further comprising the step of maintaining a flow rate of the gas of between about 2 L/min and about 12 L/min.

23. The method of reducing motion sickness of claim 14 further comprising the step of maintaining a flow rate of the gas of between about 3 L/min and 5 L/min.

24. The method of reducing motion sickness of claim 14 further comprising the step of maintaining the gas at a temperature of between about 50° F. of and about 78° F.

25. The method of reducing motion sickness of claim 14 further comprising the step of maintaining the gas at a temperature of between about 60° F. of and about 75° F.

26. The method of reducing motion sickness of claim 14 wherein the gas is introduced into the user's ear canal for between about 1 minute and about 20 minutes.

27. The method of reducing motion sickness of claim 14 wherein the gas is introduced into the user's ear canal for between about 2 minutes and about 5 minutes.

28. The method of reducing motion sickness of claim 14 wherein the gas is introduced into the user's ear canal 1 time per day for 1 to 10 consecutive days.

29. A method of reducing motion sickness comprising the steps of:
introducing a carbon dioxide gas mixture into a user's ear canal for through an earpiece worn by the user;
exhausting the gas from the user's ear canal;
maintaining a gas circulation in a user's ear canal for between about 2 minutes and about 5 minutes;
maintaining the gas at a temperature of between about 60° F. of and about 75° F.;
maintaining a flow rate of the gas of between about 3 L/min and 5 L/min;
cooling a user's tympanic membrane with the gas; and
inducing nystagmus in the user.

30. The method of reducing motion sickness of claim 29, further comprising repeating all steps of claim 29 one time per day until symptoms associated with motion sickness are reduced in the user.
